(19) United States
(12) Patent Application Publication
(10) Pub. No.: US 2014/0039975 A1
(43) Pub. Date: Feb. 6, 2014

(54) EMOTIONAL MODELING OF A SUBJECT

(71) Applicant: Sensory Logic, Inc., Minneapolis, MN (US)

(72) Inventor: Daniel A. Hill, St. Paul, MN (US)

(73) Assignee: Sensory Logic, Inc., Minneapolis, MN (US)

(21) Appl. No.: 13/828,668

(22) Filed: Mar. 14, 2013

Related U.S. Application Data
(60) Provisional application No. 61/679,540, filed on Aug. 3, 2012.
(60) Provisional application No. 61/707,600, filed on Sep. 28, 2012.
(60) Provisional application No. 61/763,826, filed on Feb. 12, 2013.

(51) Int. Cl. A61B 5/16

(52) U.S. Cl. CPC A61B 5/167
USPC 705/7.29; 702/19

(57) ABSTRACT

Systems and techniques for emotional modeling of a subject are described herein. Emotional data of a subject during exposure to a stimulus can be received. The emotional data can be interpreted to produce a result. The result can include an emotional model of the subject. The result can be presented to the user.

### CLAIM OF PRIORITY

This patent application claims the benefit of priority, under 35 U.S.C. §119(e), to U.S. Provisional Patent Application Ser. No. 61/679,540, titled “ENHANCED ATHLETE MANAGEMENT VIA EMOTION ANALYSTICS,” filed Aug. 3, 2012, U.S. Provisional Patent Application Ser. No. 61/707,600, titled “EMOTIONAL ANALYTICS VISUALIZATION,” filed Sep. 28, 2012, and U.S. Provisional Patent Application Ser. No. 61/763,826, titled “AUTOMATED PRESENT AND PREDICTIVE EMOTIONAL MODELING,” each of which is hereby incorporated by reference in its entirety.

### BACKGROUND

Underlying human emotional data can be ascertained via physiologic monitoring of a person. Examples of physiologic monitoring can include electroencephalography (EEG), galvanic skin response, temperature, and facial coding among others. Generally, the raw data is evaluated by a human coder who assigns one or more emotional components to the subject based on the monitoring. Further emotional analysis is also generally conducted by a trained professional based on the coded emotional components.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a system for emotional modeling of a subject, according to an embodiment.

FIGS. 2-14 illustrates various examples of emotional interpretation results, each according to an embodiment.

FIG. 15 illustrates an example of a method for emotional modeling of a subject, according to an embodiment.

FIG. 16 is a block diagram illustrating an example of a machine upon which one or more embodiments may be implemented.

### DETAILED DESCRIPTION

Using facial coding for physiologic monitoring is accurate and can be accomplished with relatively inexpensive and pervasive video technology. In an example, automated systems exist to interpret video of a subject (e.g., market research consumer) to identify one or more emotional components, such as anger, fear, disgust, etc. For example, video can be captured (e.g., via computer or web-cam, smart phone, tablet, etc.) and analyzed using automated facial coding. Automated facial coding can include identifying and reporting consumer facial expressions according to metrics such as the amount and timing of emotional engagement (e.g., impact), positive/negative valence, specific emotions (e.g., anger, disgust, etc.), and which emotions, if any, are dominant emotional reactions of an individual or group of subjects at any given moment in time or in a time period involving exposure to a stimulus (e.g., a product, advertisement, interview, monologue-characterized prompted or unprompted input, situation, simulation, etc.)

Automated facial coding is generally limited to determining core emotions (e.g., happiness, surprise, sadness, fear, anger, disgust, and contempt) as well as some blended emotional responses. Although these emotional components can be useful to trained persons, they are generally insufficient to allow computing systems or the untrained to draw greater conclusions as to, or act on, the emotional state of the subject. However, these emotional components can be used as parameters in emotional interpretation models of the subject at the time of the physiologic monitoring and also in predictive emotional models of the subject. These models can provide better actionable intelligence to those likely to make decisions (e.g., marketers, executives, etc.) and are also likely to lack specialized training to interpret underlying emotional data.

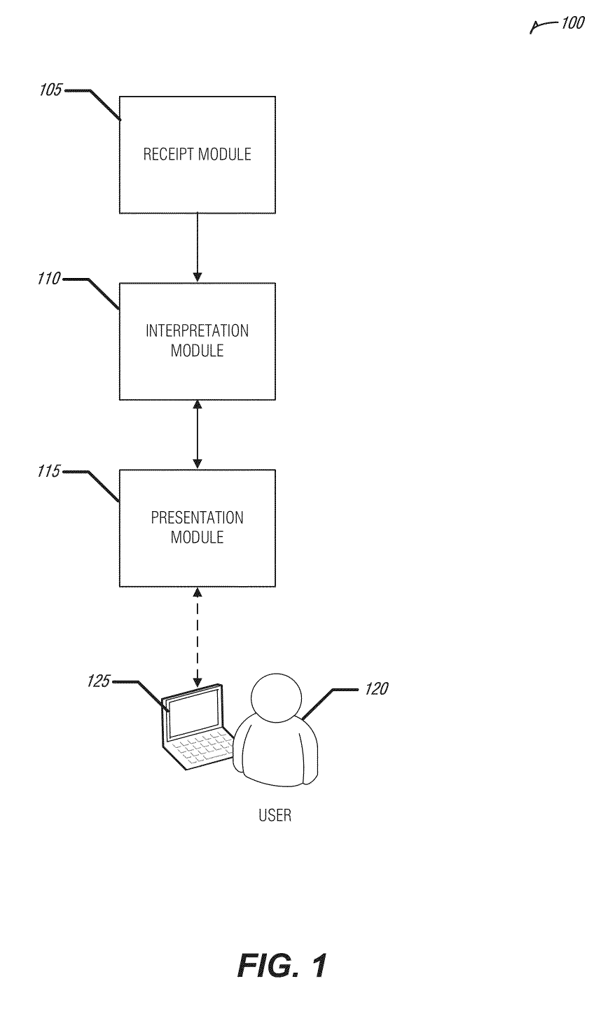

FIG. 1 illustrates an example of a system 100 for emotional modeling of a subject. The system 100 can include a receipt module 105, an interpretation module 110, and a presentation module 115. In an example, the presentation module 115 can be arranged to be communicatively coupled to a terminal 125 (e.g., a computer, mobile device, tablet, etc.).

The receipt module 105 can be arranged to receive emotional data of a subject during exposure to a stimulus. Thus, the emotional data corresponds to both the subject and the subject's exposure to the stimulus. In an example, the stimulus can be a survey (e.g., questionnaire, form, etc.). In an example, the stimulus can be a commercial item. In an example, the stimulus can be a marketing research presentation of a commercial item. In an example, the stimulus can be a survey about a commercial item.

The interpretation module 110 can be arranged to interpret the emotional data to produce a result. The result can include an emotional model of the subject. In an example, the emotional model can include an emotional segmentation for the subject. The emotional segmentation can operate like other forms of segmentation operating on demographic data, such as wealth, income, race, age, geographic location, etc. Emotional segmentation can provide, for example, another level of granularity for marketers to target product towards. Emotional segmentation can include such things as subject appeal and engagement for a commercial item (e.g., a product, service, charity, or other items upon which the subject may spend money or time). Thus, given demographically similar persons, the marketer may be able to determine that the subject likes the commercial item but is not that engaged (e.g., is not likely to act on this like).

In an example, the emotional model can include a predictive model. The predictive model can include a probability that the subject will perform an action. The predictive model can be based off of previous actions by the subject or others who correspond to the subject via the emotional data. Thus, the emotional data can be used to group the subject with others such that the actions of the others can be imputed to the subject. In an example, the predictive model can be based on a previously observed performance result corresponding to the stimulus. For example, the previously observed performance result can be the purchasing behavior of consumers to an advertisement. The emotional data can be used to classify the subject's interest and type of interest in the advertisement (e.g., great disgust). This information can be combined with previously observed purchasing behaviors of the subject with respect to this emotional data.

In an example, the emotional model can include a personality assessment. Personality assessments can provide well researched models of human behavior. A problem, however, with most personality assessments is the self-reporting nature of them. That is, for the most part, the subject answers a survey from which the assessment is derived. This participation by the subject can lead to a cognitive filtering that can introduce error into the results. By using observed emotional data, as here described, such cognitive filtering can be obviated. In an example, the personality assessment can include an assessment of Maslow's hierarchy of needs for the subject. In an example, the personality assessment can include a set of motivations for the subject. In an example, the personality assessment can include the Big Five personality traits (e.g., openness, conscientiousness, extraversion, agreeableness, and neuroticism).

In an example, the emotional model can include a say-feel gap analysis. The say-feel gap analysis can indicate a difference between the semantic meaning of a phrase (e.g., word or set of words) uttered (e.g., spoken, whispered, etc.) by the subject and a portion of the emotional data corresponding to the phrase. For example, a politician may say, “trust me, we will not close this base down,” while the emotional data indicates doubt, shame, or other emotional indicators of deceit. Thus, there is a gap between the meaning of “trust me” and the emotions of the politician.

In an example, where the stimulus is a commercial item, the emotional model can include a marketing research model. A marketing research model can indicate any of several aspects of the subject relevant to marketing a commercial item. Examples can include subject engagement with the commercial item (e.g., how interested is the subject in the commercial item?), whether the subject likes or dislikes the commercial item, what aspects of the commercial item elicit specific emotions (e.g., disgust, happiness, satisfaction, trust, etc.).

In an example, where the stimulus is a survey of a commercial item, the emotional model can include a customer satisfaction model. The customer satisfaction model can embody emotional intelligence such as customer loyalty to a product, customer frustration with a vendor process, etc. Using the observed emotional data can obviate the errors introduced by cognitive filtering when the subject completes the survey. Because retaining customers is much cheaper than attracting new customers, such a model can effectively save companies money.

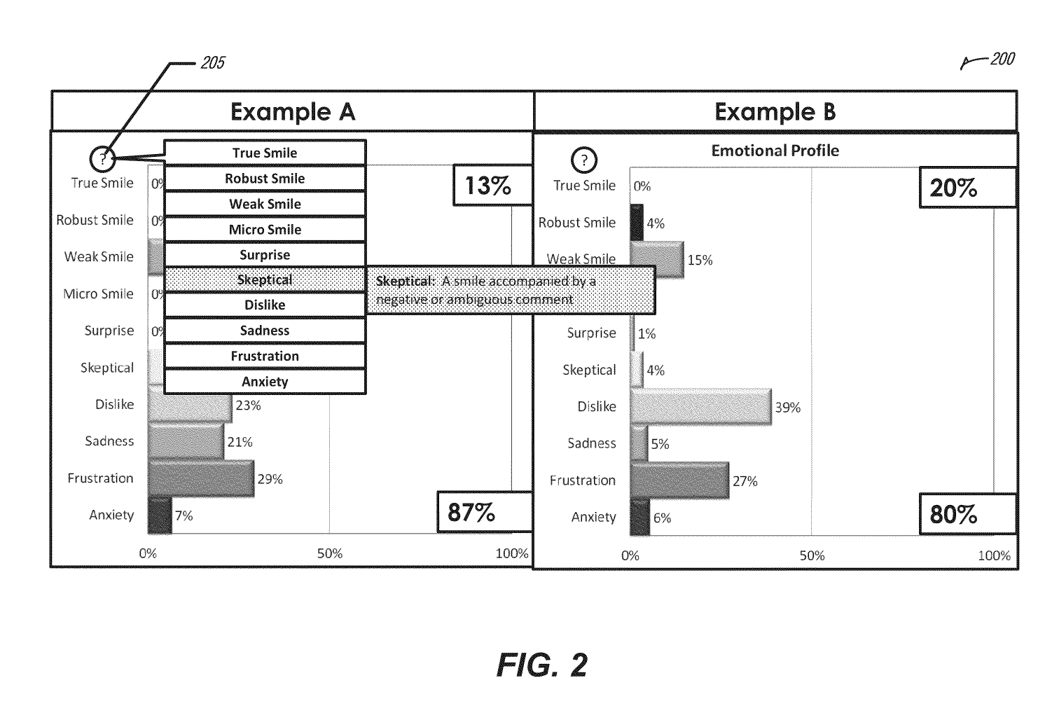

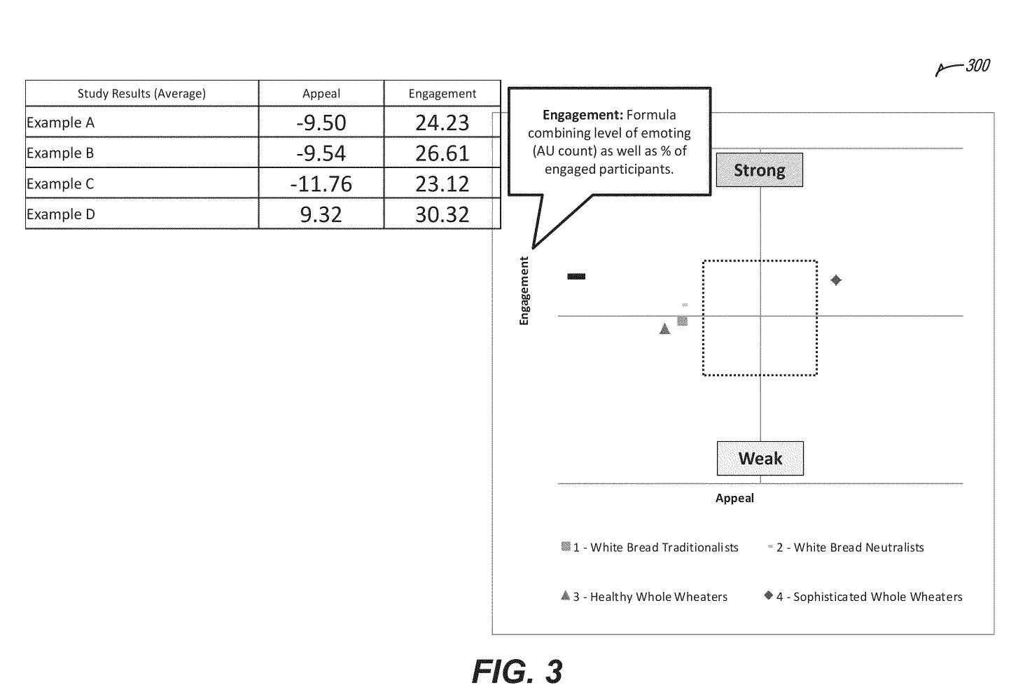

The presentation module 115 can be arranged to present the result to a user 120. In an example, the presentation module 115 can be arranged to present the result to the user 120 via the terminal 125. In an example, the presentation module 115 can be arranged to provide a user interface (e.g., on the terminal 125). The user interface can be arranged to display the result to the user 120 in a normalized manner. As discussed above, raw emotional data can be detailed and include many data points. By normalizing (e.g., standardizing) the display of such data can ease the task of the user 120 in understanding the emotional data. In an example, standardizing the result can include presenting components of the emotional data in a fixed relation to one another (e.g., emotional component 1 appears before emotional component 2). In an example, the user interface can include a drill-down element. The drill-down element can be arranged to receive a user selection of it. The drill-down element can be arranged to display, in response to receiving the selection, at least one of an underlying portion or an explanation of the emotional data used to derive the result. FIGS. 2 and 3 illustrate such a drill-down element.

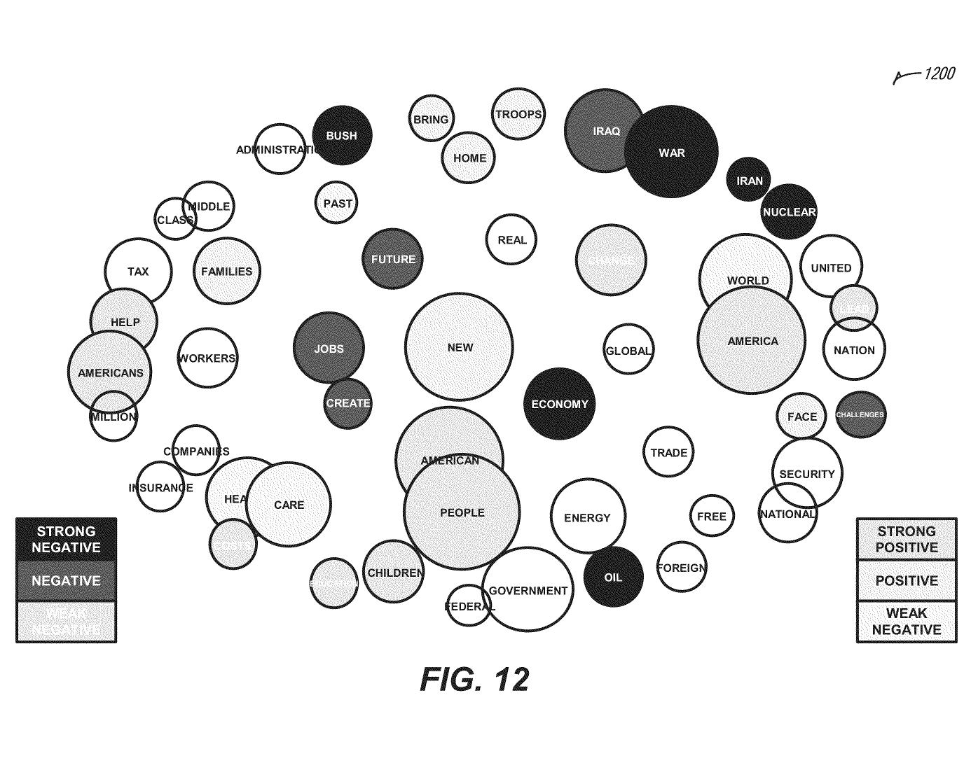

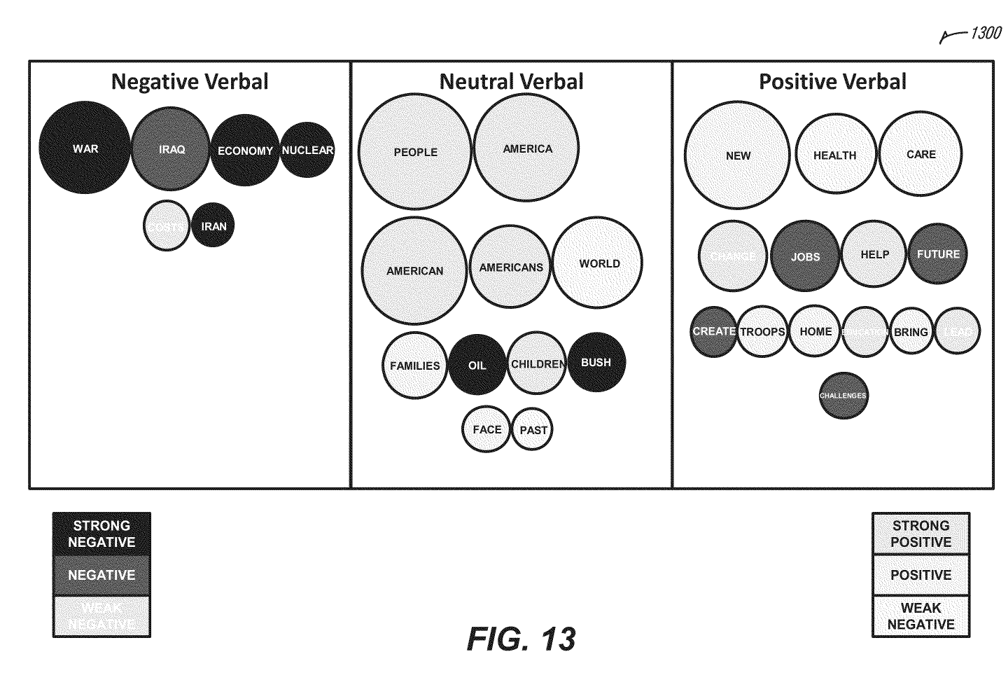

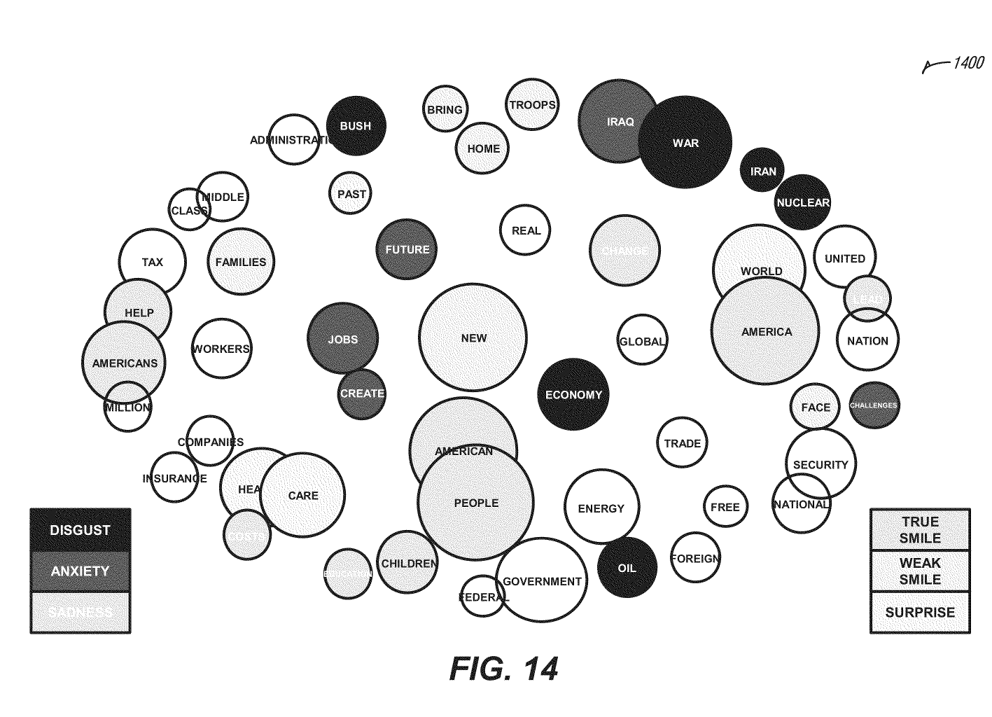

In an example, where a say-feel gap analysis exists within the result, the presentation module 115 can be arranged to provide a bubble report of the say-feel gap. Examples of such reports are illustrated in FIGS. 12-14. The bubble report can indicate engagement via the size of a bubble, the relevant phrase by printing it within the bubble, and the type of emotion via a color of the bubble. Such a representation can provide a readily understandable representation of the say-feel gap.

The features and examples described with respect to FIG. 1 provides a framework for emotional modeling of a subject. The emotional modeling can provide actionable intelligence for decision makers that is often lacking from the raw emotional data produced by coders. Several additional examples are described below and illustrated in FIGS. 2-14.

FIGS. 2-14 illustrates various examples of emotional interpretation results. The following examples include different result presentations. In order to more fully demonstrate the presentation, some sample data is used. Below are explanations for the various examples of FIGS. 2-14.

FIG. 2 illustrates a user interface 200 including a drill-down element 205. There are seven core emotions (happiness, surprise, sadness, fear, anger, disgust and contempt) as well as a number of secondary, blended emotional responses that facial coding can identify. To increase the value of the result (e.g., emotive data output), an automatic emotional interpretation system can be used to create emotional model (e.g., emotional intelligence). The emotional model can include what each emotion may mean as a response, and also integrate the output into a normative or actionable output.

In an example, the emotional model's output can be represented as a set of digital informational menus (e.g., drill-down element 205). The menus can define each emotion and emotional measurement category. In an example, a dashboard delivery device can augment or replace the menus. In an example, the menus can be created for a business context. The model can be arranged to enable the end-user to grasp how the emotive data item can be interpreted for use in identifying problems and opportunities. For example, the model can accept research indicating an unusually high level of frustration among customers who have recently visited a company's stores. The model can be arranged to determine if it is an above average level anger, to indicate what the causes of the anger are, and provide solutions to the problems (e.g., given that anger is an emotion typically caused by confusion, resistance, and/or resentment because one's value system and/or self-identity has been offended, provide solutions to mitigate these factors). Also, for example, the model can be arranged to accept data indicating a large amount of anger during exposure to a TV spot, correlate the anger to confusion to a number of rapidly changing scenes given that the number of scenes, and notify the end-user of possible solutions to the anger issue. The table below illustrates several possible components of emotional interpretation models:

| | |
|---|---|
| DEFINITIONS | Appeal |
| | Big Five Traits |
| | Emotions |
| | Engagement |
| | Motivations |
| EXPLANATIONS | Criteria for ranking tables |
| | Levels of coding (silver, gold, platinum) |
| | Overall emotion pie chart categories |
| | Second-by-second dots |
| | Sums of positive/negative emotions in profile |
| EXAMPLES | Anecdotes from similar previous tests |
| | Learnings for About Face, Emotionomics |
| | Video clips of subjects |
| CONTEXT | Normative data |
| | Second-by-second context from About Face |
| | Stimuli-specific context from different |
| | dominant emotions |

For example, the models can include definitions of high-level output, such as Appeal (e.g., did the subject like or dislike something possible including the degree of the like or dislike), Big Five personality traits, emotions, engagement (e.g., to what degree the subject is emoting), or subject motivations. The models can also include explanations to end-users to allow those end-users to make greater use of the models' output. The models can be based on examples or context of the subject or the stimulus. FIG. 3 illustrates an example output of an emotional interpretation model indicating engagement and appeal.

In addition to providing emotional definitions, a second interface (e.g., menu) can details a set of consumer needs, wants, feared risks, or universal motivations that can both be defined and put into a business-minded interpretative context. A wide variety of options are available for modeling such needs, wants, risks and universal motivations. Some examples can include modeling functions based on: Maslow's hierarchy of needs; Driven (Nohria, Lawrence), including key motivations of defend, learn, bond and acquire; Motivations identified by Northwestern University Professor, Andrew Ortony. An emotive template can be arranged, for example, to evaluate a branded offer that is meant to address a product, or a TV spot, and determine, from an emotive point of view, how well the product or commercial does in addressing a set of motivations. In an example, the set of motivations can be ranked in priority order regarding degree of importance. Motivations (e.g., needs, wants, risks, or benefits sought by) of the target market (e.g., potential end-users) can be pre-determined through interviews or surveys. Emotive data can be used to verify a degree of successful fulfillment of needs/wants and avoidance of risks being measured and acted on in terms of potential business initiatives to improve the outcome (e.g., produce more sales). The same will go for benefits successfully realized, as denoted through the emotive data, and for barriers to acceptance, endorsement, consideration or persuasion. Again, all results can also be placed into a normative context to signal optimal, average and sub-optimal performance.

In an example of an automated emotional interpretation system, emotional interpretation categories like appeal, engagement, or the like can be used to provide actionable output for end-users. In an example, an interface (e.g., pull-down menu option) can place the emotive results into the context of a predictive formula for modeling purposes.

Other interfaces (e.g., menus that could also be pulled down or otherwise actuated) can be used to access the following: a) what each emotion means in terms of behavior; b) what each emotion means in terms of level of acceptance (of advertising, product, etc.); c) what each emotion means relative to acting on a call to action; d) what each emotion means in terms of likelihood of recall; e) what each emotion means in terms of outcome orientation; f) what each emotions means in terms of level of attention; g) what each emotion means in terms of risk tolerance; h) what each emotion means in terms of decision making; i) what each type of facial coding means and how it is performed; j) video examples of emotional response during verbal feedback; k) normative data relative to the category of the stimulus, etc.

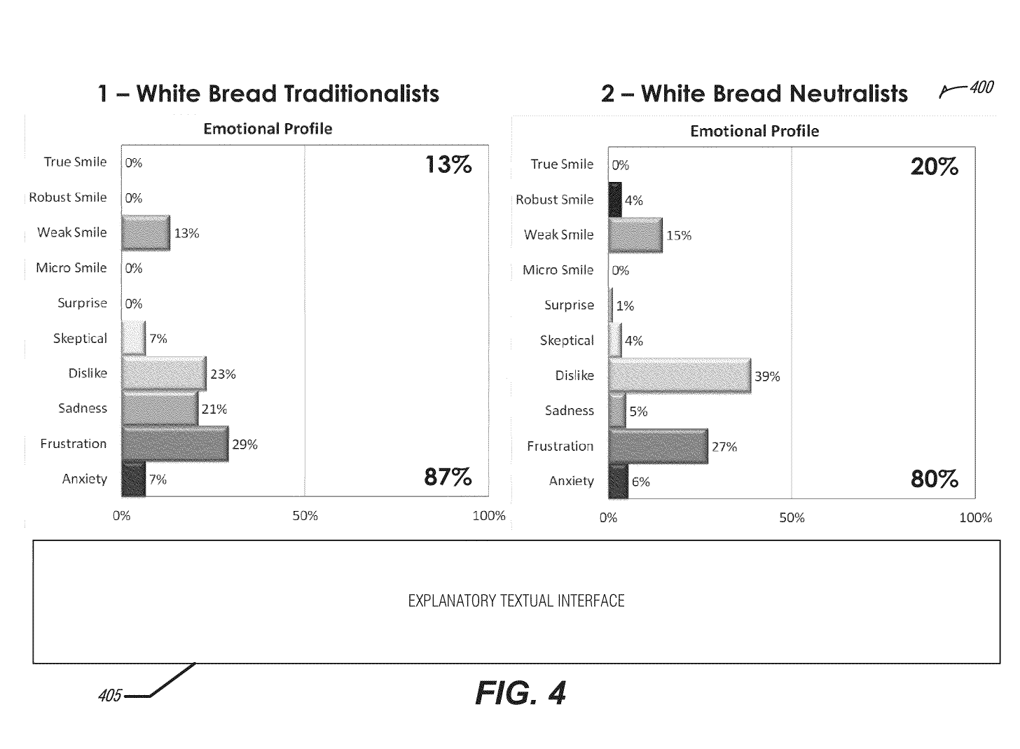

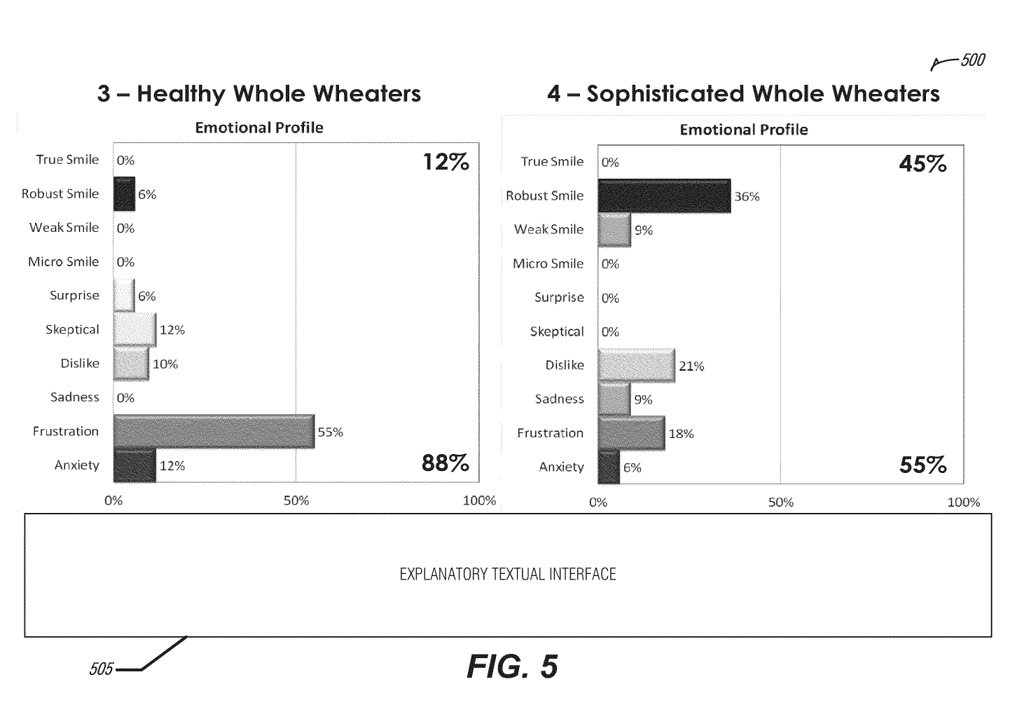

FIGS. 4 and 5 generally relate to emotional segmentation. Often business plans (e.g., marketing segmentation schemes) are rooted in factors such as consumer demographics, purchase frequency, profit margin, etc. Physiological monitoring for emotional response (e.g., facial coding) can be used to collect emotive data and to reach a higher level of understanding subjects and, therefore, efficiency in tailoring objectives towards those subjects. For example, to know who one is selling to on a level of intimate emotional understanding can afford a serious competitive advantage. Consider the case of a packaged goods company who wants to know how best to arrest the fall in sales of a food item by hitting the right motivational and emotive buttons. The solution can be based on knowing more intimately who the customers really are, including how those customers relate to the business issue at hand. For example, if the food item has nutritional shortcomings but good taste, a segment of customers that want to feel in control of their destiny in terms of what kind of nutritional ingredients go into their bodies may index high on anger, while a group who simply loves the food item based on taste alone may index high on happiness, as a sign of pleasure. Knowing the specific emotional mix of an individual or targeted group of consumers may cause the marketing effort to be more successful.

FIG. 4 illustrates categorizations of consumers (e.g., 1—White Bread Traditionalists and 2—White Bread Neutralists) as well as the respective emotive data for these categories. Additionally, the output illustrated in FIG. 4 includes an explanatory textual interface explaining to the layman the implications of various portions of the emotive data. FIG. 5 illustrates a similar categorization to that illustrated in FIG. 4 for a slightly different product (e.g., wheat bread instead of white bread).

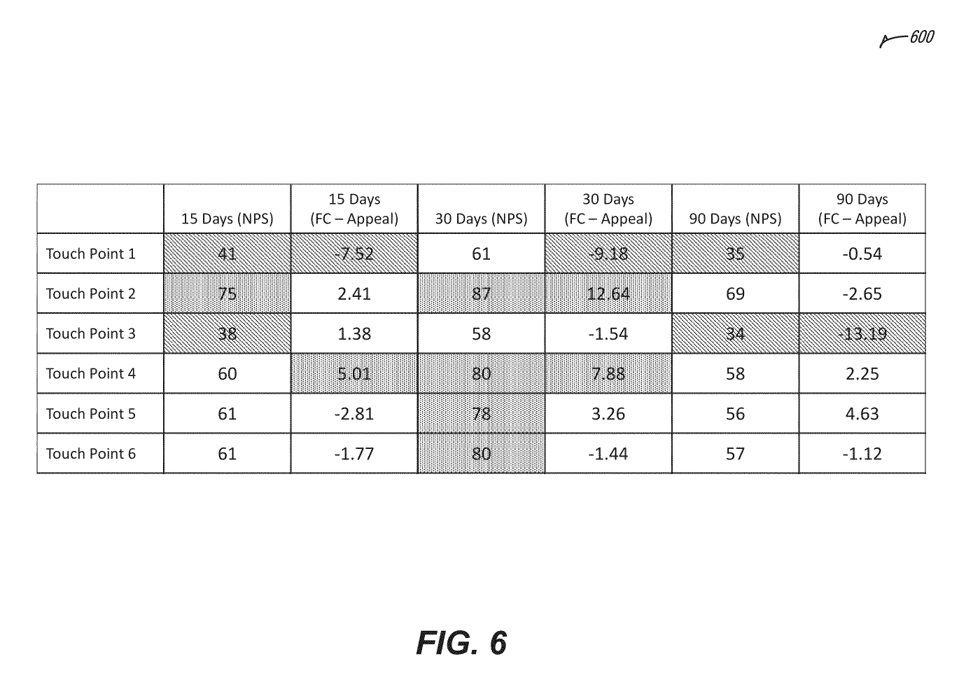

FIG. 6 generally relates to customer satisfaction modeling. Customer satisfaction is often a business goal that can be based in emotional understanding of customers. In an example, customer satisfaction can be determined via a brief customer survey card at the end of a business transaction. Whether the answers are honest, and whether they get skewed by having only angry (e.g., thus being highly-motivated) customers bother to fill out the surveys is among the concerns or liabilities of such an approach. In an example, customer satisfaction can be determined using the Net Promoter Score (NPS). NPS can include taking a 10-point satisfaction scale and assuming that a score of 9 or 10 in terms of degree of satisfaction with a company translates into that person being a “promoter” or endorser of the company. To guard against “lip service” grade inflation, a score of 6 or below is assumed to mean the same person taking the survey is actually a detractor or person who dislikes the company, isn't loyal, and may in fact harm the company by sharing that dissatisfaction with others, hence a business risk. The NPS score is derived from subtracting the number of detractors from the number of promoters, as a percentage of subjects who take the survey.

However, nothing in such an approach delivers a non-cognitively filtered emotive component, which can be an important factor in customer satisfaction because loyalty, in the end, is a feeling, a sense of belonging or wanting to belong to a brand as a customer or as an unpaid, spontaneous advocate for a company's products and services. In an example, NPS can be augmented to include facial coding and, in an example, a degree of latency. Improving NPS can provide direction for a company or institution to model consumer satisfaction levels, identify areas of practice where improvements could be made to ensure loyalty, grow market share (e.g., by simultaneously attracting and retaining customers), and ultimately growing profitability.

An example satisfaction-to-loyalty monitoring program can be arranged to accept input data from a variety of sources, such as, web-based, web-cam enabled surveys, to telephonic and video conferencing interviews, contact center (like a call center phone bank), central location, trade show, in home, mobile ethnography, mobile panels, focus groups, video conferences, mobile device enabled input (e.g., embedding survey links in mobile apps), and on-premise intercept interviews (including those operated automatically, with an unmanned but programmed video station). In an example, video surveys can be used. While some surveys may take as long as 3 to 5 minutes to complete, thus driving down participation rates and causing “brown-out” suspect answers or input, such a system using video that gets facially coded may take less than a minute to record.

With the automatic interpretation options delineated earlier, collected data (e.g., including facially coding augmented survey data) can be modeled and readily understood by people within a company from high to low in terms of rank and level of analytical capacity. Using textual analytics to identify key words, themes, and categories and marrying such analytics to the emotions associated with those word choices, it becomes possible to learn the root causes of satisfaction or dissatisfaction, and how it might be leveraged or resolved depending on valence. That determination can then, in turn, be shared with the operative department, relevant staff members, and the like, to make the optimal modifications in stimuli, procedures, and the like. On a granular basis, such a video-enabled version of NPS can result in gaining emotional data that serves, for instance, as an emotive audit of the performance of an individual sales person meeting with a series of clients, or a financial advisor interacting with prospects and clients, or a crew of clerks in a store helping customers.

As a result, how to better lure new customers and build on practices that create promoters can be enacted, as well as making adjustments to save a customer in distress and thus at risk of being “lost” to the company (“customer recovery”). Goals and benchmarks against which performance and the emotive dynamics of customers interfacing with the company can be henceforth monitored on an on-going basis to lift company performance and profitability. Outputs to serve that goal could include performance heat maps that cite where opportunities and threats, from a customer emotive viewpoint, exist as well as changes in emotive performance level or satisfaction level within the past 30 days, or 90 days, for example, by customer touch point, including specific examples like a particular store location, service department branch, sales representative, etc. Knowing which touch points also generate the largest comparative amount of emotive data could be essential to knowing where a company should place its focus; given that those touch points generating more data can be taken as of greatest importance to customer satisfaction. This emotive data could be provided to all system users within a company, regarding those deemed responsible for monitoring and improving customer satisfaction, including in a specific NPS context. FIG. 6 illustrates an example output correlating touch points to different parameters. Also represented are diagonally shaded and shaded cells, respectively signaling, for example, bad results and good results to enable quick identification by the user 120.

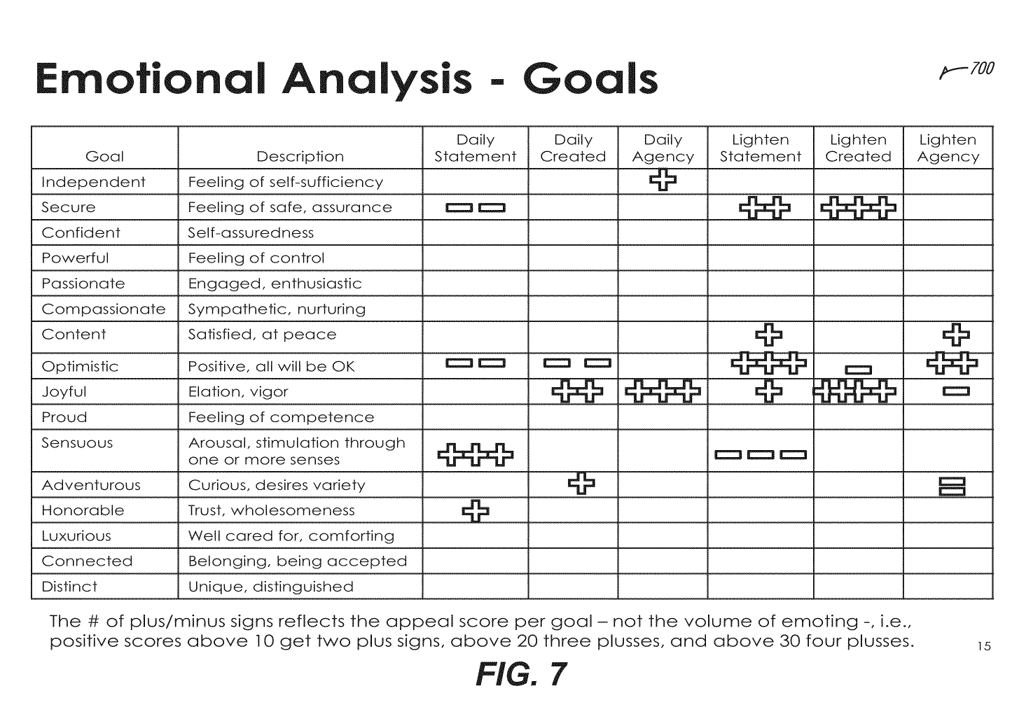

FIGS. 7-11 illustrate examples of results 700, 800, 900, 1000, and 1100. FIG. 7 includes a goal based model for the subject. The result 700 includes several visual features to model the underlying emotional data in a way that is easily accessible to a layman.

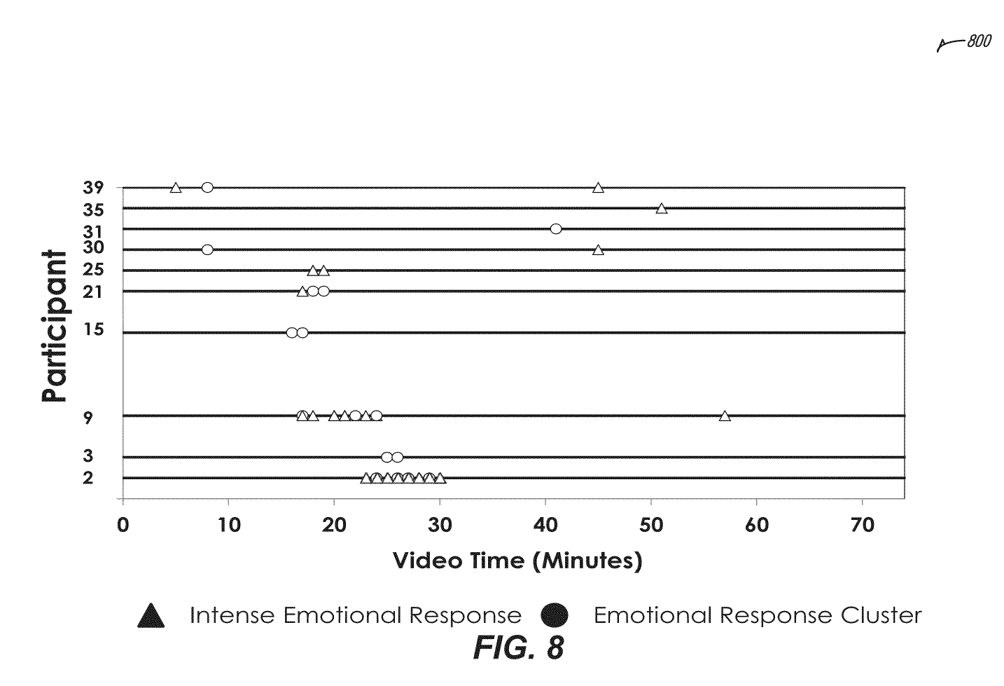

The result 800, illustrated in FIG. 8, includes a multiple subject emotional model for a stimulus (e.g., video). The result 800 models the underlying emotional data as an intense emotional response or a cluster of emotional response for each subject over time in the video.

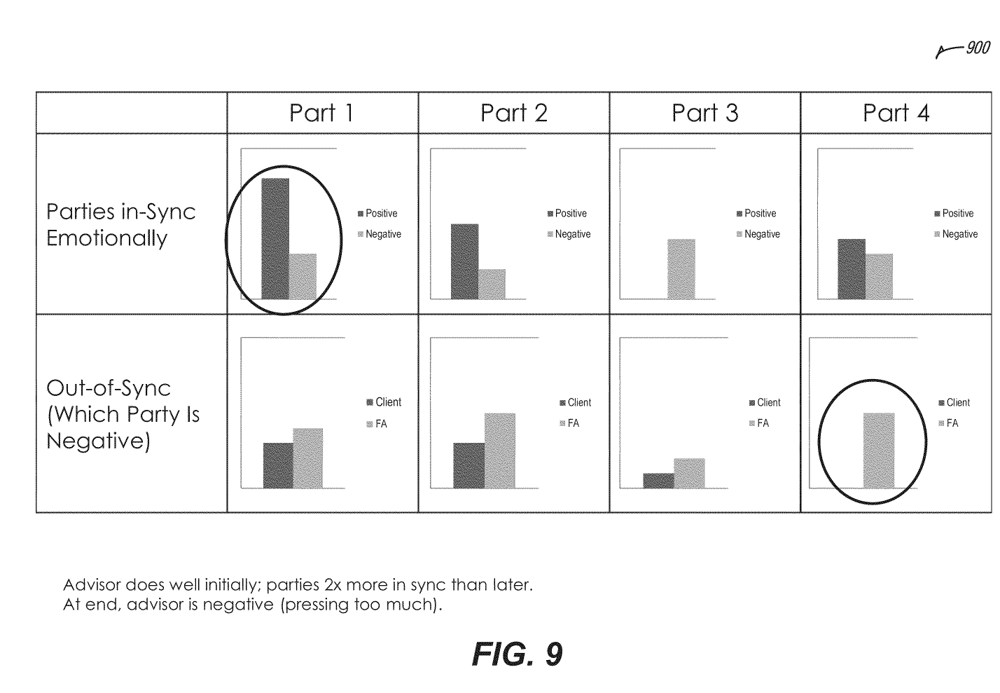

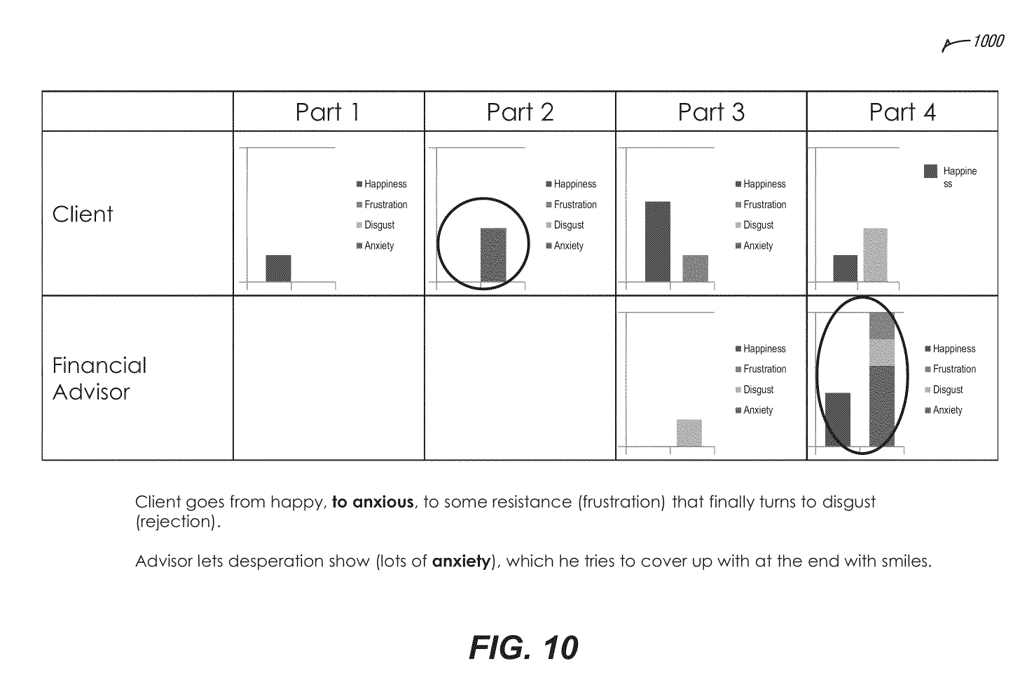

The results 900 and 1000, respectively illustrated in FIGS. 9 and 10, include an emotional model of subjects client and financial advisor over several discrete parts of their interaction. The model simplifies the emotional analysis to demonstrate how in sync the parties are, who is having a problem, and what emotions are being expressed by the parties.

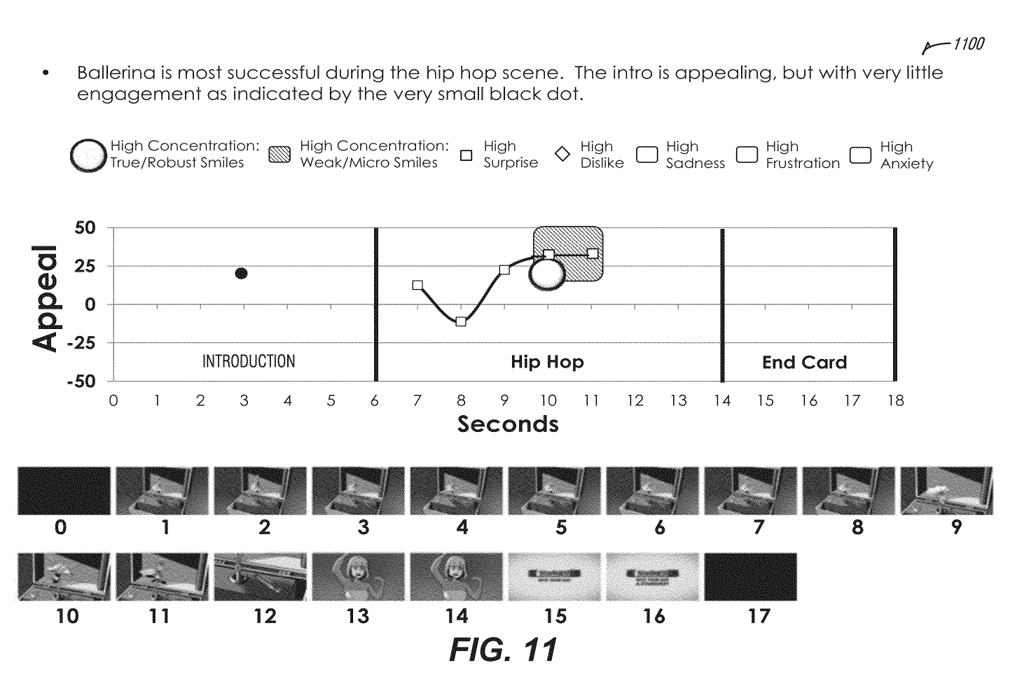

The result 1100, illustrated in FIG. 11, includes both the emotional model and the stimulus (e.g., scenes of a video) from which the underlying emotional data was collected. The result 1100, indicates the type of emotional data via shape and color, the amount of engagement by the size of the shape, and also correlates the shapes to subject appeal as well as the position in the video of a scene that elicited the subject response. The above illustrated examples of emotional modeling can produce more actionable intelligence to principle users 120, such as marketers, than the raw underlying emotional data.

FIGS. 12-14 illustrate several examples of say-feel gap bubble reports. These bubble reports are also known as word clouds. The process of integrating emotional and verbal output is another opportunity for additional efficiency gains. In an example, emotional output can be manually inserted into transcriptions (e.g., by comparing timestamps and reviewing the video files). In an example, verbal and emotional integration can be accomplished by using a computing device to perform one or more operations in the task. Operation one can include automatically copying an audio track from videos that need to be transcribed. These copied audio files can be queued for automatic transcription by voice-recognition system. In an example, the voice-recognition system can retain, via metadata, the beginning and ending time of each word spoken (e.g., in MM:SS.SSS format). This timing can be used later in the process for correlating emotional data to the individual words.

In an example, verbal timing metadata can be used to insert (e.g., correlate) emotional output observed during the facial coding process. For example, action unit (AU) numbers input during the facial coding process can be automatically replaced with the corresponding emotions (e.g., interrupted emotions based on the observed AUs) when entered into the transcript. An emotion entered into the transcript can automatically re-color text of emotion names according to their respective valence (e.g., blue for positive emotions and red for negative emotions.)

In an example, the results produced above can be used with textual analysis software to combine the verbal/emotional results into graphical output. Word clouds can illustrate, either separately or in the same graphic, the proximity of words as they occurred within the text, the valence of said words, the say/feel gap of verbal output versus the corresponding emotional response and dominant emotions corresponding to verbal output, as illustrated in FIGS. 12-14.

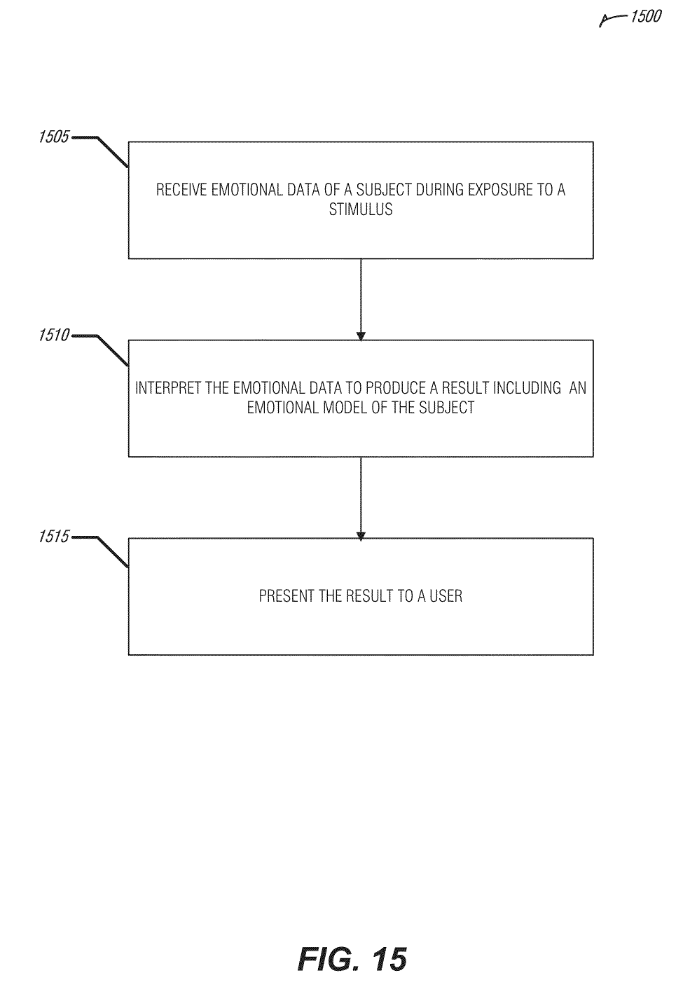

FIG. 15 illustrates an example of a method 1500 for emotional modeling of a subject.

At operation 1505 emotional data of a subject curing exposure to a stimulus can be received. In an example, the stimulus can be a survey. In an example, the survey can pertain to a commercial item. In an example, the stimulus can be a commercial item.

At operation 1510 the emotional data can be interpreted to produce a result including an emotional model of the subject. In an example, the emotional model can include an emotional segmentation for the subject. In an example, the emotional model can include a predictive model. The predictive model can include a probability that the subject will perform an action. In an example, the predictive model can be based on a previously observed performance result corresponding to the stimulus. In an example, the emotional model can include a personality assessment. In an example, the personality assessment can include the Big Five personality traits. In an example, the emotional model can include a say-feel gap analysis. The say-feel gap analysis can indicate a difference between the semantic meaning of a phrase uttered by the subject and a portion of the emotional data corresponding to the phrase. In an example, where the stimulus is a commercial item, the emotional model can include a marketing research model. In an example, where the stimulus is a survey about a commercial item, the emotional model can include a customer satisfaction model.

At operation 1515 the result can be presented to a user. In an example, presenting the result to the user can include providing a user interface arranged to display the result in a normalized manner. In an example, the user interface can include a drill-down element. The drill-down element can be arranged to receive a user selection. The drill-down element can be arranged to display, in response to the user selection, an underlying portion of the emotional data used to derive the result. In an example, where the emotional model includes a say-feel gap analysis, presenting the result can include a bubble report of the say-feel gap analysis.

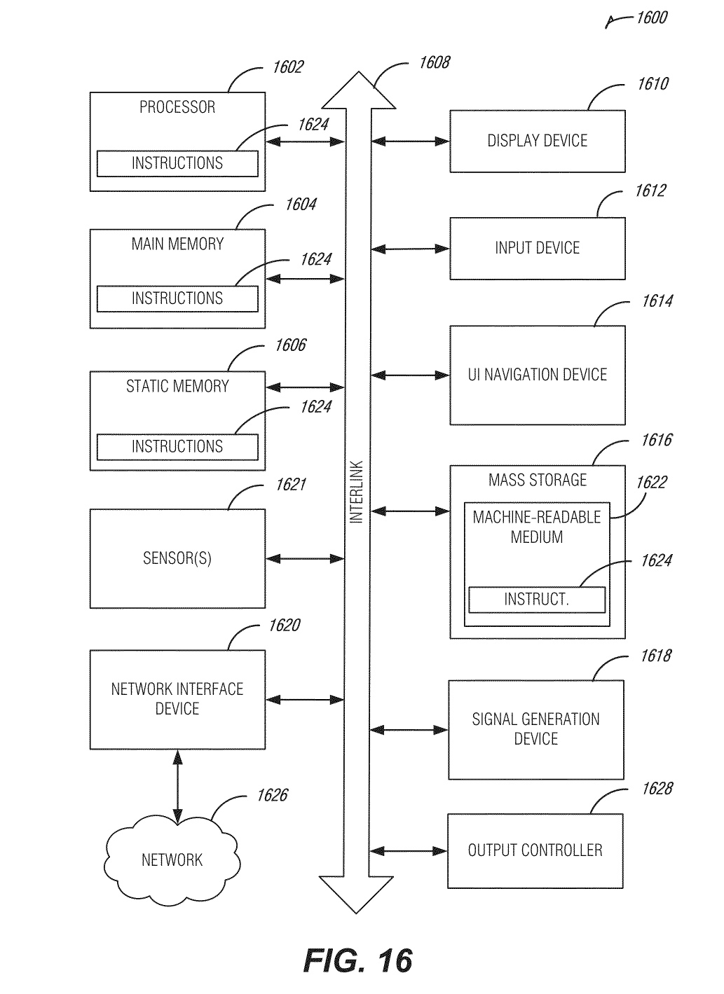

FIG. 16 illustrates a block diagram of an example machine 1600 upon which any one or more of the techniques (e.g., methodologies) discussed herein may perform. In alternative embodiments, the machine 1600 may operate as a standalone device or may be connected (e.g., networked) to other machines. In a networked deployment, the machine 1600 may operate in the capacity of a server machine, a client machine, or both in server-client network environments. In an example, the machine 1600 may act as a peer machine in peer-to-peer (P2P) (or other distributed) network environment. The machine 1600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine readable medium. In an example, the software, when executed by the underlying hardware of the module, causes the hardware to perform the specified operations.

Accordingly, the term “module” is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

Machine (e.g., computer system) 1600 may include a hardware processor 1602 (e.g., a central processing unit (CPU), a graphics processing unit (GPU), a hardware processor core, or any combination thereof), a main memory 1604 and a static memory 1606, some or all of which may communicate with each other via an interlink (e.g., bus) 1608. The machine 1600 may further include a display unit 1610, an alphanumeric input device 1612 (e.g., a keyboard), and a user interface (UI) navigation device 1614 (e.g., a mouse). In an example, the display unit 1610, input device 1612 and UI navigation device 1614 may be a touch screen display. The machine 1600 may additionally include a storage device (e.g., drive unit) 1616, a signal generation device 1618 (e.g., a speaker), a network interface device 1620, and one or more sensors 1621, such as a global positioning system (GPS) sensor, compass, accelerometer, or other sensor. The machine 1600 may include an output controller 1628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1616 may include a machine readable medium 1622 on which is stored one or more sets of data structures or instructions 1624 (e.g., software) embodying or utilized by any one or more of the techniques or functions described herein. The instructions 1624 may also reside, completely or at least partially, within the main memory 1604, within static memory 1606, or within the hardware processor 1602 during execution thereof by the machine 1600. In an example, one or any combination of the hardware processor 1602, the main memory 1604, the static memory 1606, or the storage device 1616 may constitute machine readable media.

While the machine readable medium 1622 is illustrated as a single medium, the term “machine readable medium” may include a single medium or multiple media (e.g., a centralized or distributed database, and/or associated caches and servers) configured to store the one or more instructions 1624.

The term “machine readable medium” may include any medium that is capable of storing, encoding, or carrying instructions for execution by the machine 1600 and that cause the machine 1600 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine readable medium comprises a machine readable medium with a plurality of particles having resting mass. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1624 may further be transmitted or received over a communications network 1626 using a transmission medium via the network interface device 1620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1620 may include one or more physical jacks (e.g., Ethernet, coaxial, or phone jacks) or one or more antennas to connect to the communications network 1626. In an example, the network interface device 1620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1600, and includes digital or analog communications signals or other intangible medium to facilitate communication of such software.

### ADDITIONAL NOTES & EXAMPLES

Example 1 can include subject matter subject matter (such as a method, means for performing acts, or machine readable medium including instructions that, when performed by a machine cause the machine to performs acts) comprising receiving emotional data of a subject during exposure to a stimulus, interpreting the emotional data to produce a result—the result including an emotional model of the subject, and presenting the result to a user.

In Example 2, the subject matter of Example 1 can optionally include, wherein the emotional model includes an emotional segmentation for the subject.

In Example 3, the subject matter of any of Examples 1-2 can optionally include, wherein the emotional model includes a predictive model—the predictive model including a probability that the subject will perform an action.

In Example 4, the subject matter of Example 3 can optionally include, wherein the predictive model is based on a previously observed performance result corresponding to the stimulus.

In Example 5, the subject matter of any of Examples 1-4 can optionally include, wherein the emotional model includes a personality assessment.

In Example 6, the subject matter of Example 5 can optionally include, wherein the personality assessment includes the Big Five personality traits.

In Example 7, the subject matter of Example 6 can optionally include, wherein the stimulus is a survey.

In Example 8, the subject matter of any of Examples 1-7 can optionally include, wherein presenting the result to the user includes providing a user interface—the user interface arranged to display the result in a normalized manner.

In Example 9, the subject matter of Example 8 can optionally include, wherein the user interface includes a drill-down element—the drill-down element arranged to receive a user selection and display an underlying portion of the emotional data used to derive the result in response to receiving the selection.

In Example 10, the subject matter of Examples 1-9 can optionally include, wherein the emotional model includes a say-feel gap analysis—the say-feel gap analysis indicating a difference between the semantic meaning of a phrase uttered by the subject and a portion of the emotional data corresponding to the phrase.

In Example 11, the subject matter of Example 10 can optionally include, wherein presenting the result includes a bubble report of the say-feel gap.

In Example 12, the subject matter of any of Examples 1-11, wherein the stimulus is a commercial item—and wherein the emotional model is a marketing research model.

In Example 13, the subject matter of any of Examples 1-12 can optionally include, wherein the stimulus is a survey about a commercial item—and wherein the emotional model is a customer satisfaction model.

Example 14 can include, or can optionally be combined with the subject matter of any one of Examples 1-22 to include, subject matter such as (such as a device, apparatus, or network interface device for emotional modeling of a subject) comprising a receipt module arranged to receive emotional data of a subject during exposure to a stimulus, an interpretation module arranged to interpret the emotional data to produce a result—the result including an emotional model of the subject, and a presentation module arranged to present the result to a user.

In Example 15, the subject matter of Example 14 can optionally include, wherein the emotional model includes an emotional segmentation for the subject.

In Example 16, the subject matter of any of Examples 14-15 can optionally include, wherein the emotional model includes a predictive model—the predictive model including a probability that the subject will perform an action.

In Example 17, the subject matter of Example 16 can optionally include, wherein the predictive model is based on a previously observed performance result corresponding to the stimulus.

In Example 18, the subject matter of any of claims 14-17 can optionally include, wherein the emotional model includes a personality assessment.

In Example 19, the subject matter of Example 18 can optionally include, wherein the personality assessment includes the Big Five personality traits.

In Example 20, the subject matter of Examples 19 can optionally include, wherein the stimulus is a survey.

In Example 21, the subject matter of any of Examples 14-20 can optionally include, wherein to present the result to the user includes the presentation module arranged to provide a user interface—the user interface arranged to display the result in a normalized manner.

In Example 22, the subject matter of Example 21 can optionally, wherein the user interface includes a drill-down element—the drill-down element arranged to receive a user selection and display an underlying portion of the emotional data used to derive the result in response to receiving the selection.

In Example 23, the subject matter of any of claims 14-22 can optionally include, wherein the emotional model includes a say-feel gap analysis—the say-feel gap analysis indicating a difference between the semantic meaning of a phrase uttered by the subject and a portion of the emotional data corresponding to the phrase.

In Example 24, the subject matter of Example 23 can optionally include, wherein to present the result includes the presentation module arranged to provide a bubble report of the say-feel gap.

In Example 25, the subject matter of any of claims 14-24 can optionally include, wherein the stimulus is a commercial item—and wherein the emotional model is a marketing research model.

In Example 26, the subject matter of any of claims 14-25 can optionally include, wherein the stimulus is a survey about a commercial item, and wherein the emotional model is a customer satisfaction model.

The above detailed description includes references to the accompanying drawings, which form a part of the detailed description. The drawings show, by way of illustration, specific embodiments that may be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is to allow the reader to quickly ascertain the nature of the technical disclosure, for example, to comply with 37 C.F.R. §1.72(b) in the United States of America. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A machine-readable medium including instructions that, when executed by a machine, cause the machine to preform operations comprising:
receiving emotional data of a subject during exposure to a stimulus;
interpreting, using a hardware processor, the emotional data to produce a result, the result including an emotional model of the subject; and
presenting the result to a user.

2. The machine-readable medium of claim 1, wherein the emotional model includes an emotional segmentation for the subject.

3. The machine-readable medium of claim 1, wherein the emotional model includes a predictive model, the predictive model including a probability that the subject will perform an action.

4. The machine-readable medium of claim 1, wherein the emotional model includes a personality assessment.

5. The machine-readable medium of claim 1, wherein presenting the result to the user includes providing a user interface, the user interface arranged to display the result in a normalized manner.

6. The machine-readable medium of claim 5, wherein the user interface includes a drill-down element, the drill-down element arranged to receive a user selection and display an underlying portion of the emotional data used to derive the result in response to receiving the selection.

7. The machine-readable medium of claim 1, wherein the emotional model includes a say-feel gap analysis, the say-feel gap analysis indicating a difference between a semantic meaning of a phrase uttered by the subject and a portion of the emotional data corresponding to the phrase.

8. The machine-readable medium of claim 1, wherein the stimulus is a commercial item, and wherein the emotional model is a marketing research model.

9. A method comprising:
receiving emotional data of a subject during exposure to a stimulus;
interpreting, using a hardware processor, the emotional data to produce a result, the result including an emotional model of the subject; and
presenting the result to a user.

10. The method of claim 9, wherein the emotional model includes an emotional segmentation for the subject.

11. The method of claim 9, wherein the emotional model includes a predictive model, the predictive model including a probability that the subject will perform an action.

12. The method of claim 9, wherein the emotional model includes a personality assessment.

13. The method of claim 9, wherein presenting the result to the user includes providing a user interface, the user interface arranged to display the result in a normalized manner.

14. The method of claim 13, wherein the user interface includes a drill-down element, the drill-down element arranged to receive a user selection and display an underlying portion of the emotional data used to derive the result in response to receiving the selection.

15. The method of claim 9, wherein the emotional model includes a say-feel gap analysis, the say-feel gap analysis indicating a difference between a semantic meaning of a phrase uttered by the subject and a portion of the emotional data corresponding to the phrase.

16. The method of claim 9, wherein the stimulus is a commercial item, and wherein the emotional model is a marketing research model.

17. A system comprising:
a receipt module arranged to receive emotional data of a subject during exposure to a stimulus;
an interpretation module arranged to interpret the emotional data to produce a result, the result including an emotional model of the subject; and
a presentation module arranged to present the result to a user.

18. The system of claim 17, wherein the emotional model includes an emotional segmentation for the subject.

19. The system of claim 17, wherein the emotional model includes a predictive model, the predictive model including a probability that the subject will perform an action.

20. The system of claim 17, wherein the emotional model includes a personality assessment.

21. The system of claim 17, wherein to present the result to the user includes the presentation module arranged to provide a user interface, the user interface arranged to display the result in a normalized manner.

22. The system of claim 21, wherein the user interface includes a drill-down element, the drill-down element arranged to receive a user selection and display an underlying portion of the emotional data used to derive the result in response to receiving the selection.

23. The system of claim 17, wherein the emotional model includes a say-feel gap analysis, the say-feel gap analysis indicating a difference between a semantic meaning of a phrase uttered by the subject and a portion of the emotional data corresponding to the phrase.

24. The system of claim 17, wherein the stimulus is a commercial item, and wherein the emotional model is a marketing research model.
